(19) United States
(12) Patent Application Publication
(10) Pub. No.: US 2005/0136027 A1
(43) Pub. Date: Jun. 23, 2005

(54) Cosmetic hair composition based on isoeicosane and on a silicone fixing polyurethane

(76) Inventors: Sabine Vrignaud, La Plaine St. Denis (FR); Emmanuelle Belli, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/868,861

(22) Filed: Jun. 17, 2004

Related U.S. Application Data
(60) Provisional application No. 60/505,166, filed on Sep. 24, 2003.

(30) Foreign Application Priority Data
Jun. 17, 2003 (FR) 0307289

(51) Int. Cl. A61K 7/06; A61K 7/11

(52) U.S. Cl. 424/70.122; 424/70.17

(57) ABSTRACT

The present disclosure relates to a cosmetic hair composition, comprising, in a cosmetically acceptable medium, isoeicosane and at least one silicone fixing polyurethane. It is also directed towards a cosmetic process, such as a process for fixing and/or holding a hairstyle using said composition, and for giving the hair a sheen.

This application claims benefit of U.S. Provisional Application No. 60/505,166, filed Sep. 24, 2003.

The present disclosure relates to novel cosmetic hair compositions comprising isoeicosane and at least one silicone fixing polyurethane. It also relates to a cosmetic process, for instance, a process for fixing and/or holding the hairstyle using the compositions disclosed herein, and also for giving the hair a sheen.

It is known to use isoeicosane, a nonvolatile hydrocarbon-based oil, as a conditioning agent in cosmetic hair compositions. For example, patent application FR 2,815,853 describes cosmetic hair compositions in the form of a mousse and comprising at least one aminoplast backbone polymer, with isoeicosane as a possible conditioning agent.

However, there is a need for hair products that can make it possible to give the hair some hold and a long lasting appearance of sheen, without making it too markedly greasy in nature. Such properties are not necessarily provided by the compositions of the state of the art.

Thus, the present disclosure proposes to provide a solution to this deficiency.

For instance, the present inventors have found that compositions comprising isoeicosane and at least one silicone fixing polyurethane may not exhibit all the drawbacks indicated above, and can, for example, fix the hairstyle very well, while providing good cosmetic properties. For example, these compositions can adhere well to the hair and can, for instance, make the hair smooth to the touch. It has also been noted that these compositions can give the hair an intense and persistent sheen. Finally, the compositions as disclosed herein can be formulated in alcoholic medium.

Other aspects of the present disclosure will emerge on reading the description and examples which follow.

One aspect of the present disclosure is therefore a cosmetic hair composition, comprising, in a cosmetically acceptable medium, isoeicosane and at least one silicone fixing polyurethane.

Another aspect of the present disclosure concerns a cosmetic hair process, for instance, a process for fixing and/or holding a hairstyle using the compositions as disclosed herein.

Still another aspect of the present disclosure is the use of the compositions disclosed herein for giving the hair a sheen.

Isoeicosane is a hydrocarbon which is liquid at ambient temperature. It may for example, be the commercial product sold under the name PERMETHYL 102 A by the company Bayer.

The isoeicosane may be present in the composition in an amount, for example, ranging from 0.05 to 20% by weight, relative to the total weight of the composition, such as ranging from 1 to 10% of the total weight of the composition.

For the purpose of the present disclosure, the term “fixing polymer” means any polymer which makes it possible to give shape to the hair or to maintain the shape.

The silicone fixing polyurethanes of the present disclosure may be chosen from amphoteric, anionic and nonionic polyurethanes; for example, they may be chosen from anionic and non-ionic polyurethanes. Such polymers are, for example, described in French Patent Nos. FR 2,708,199 and FR 2,743,297.

By way of non-limiting example, the silicone fixing polyurethane may be made up of an arrangement of blocks, this arrangement may be obtained for instance from:
(1) at least one compound that comprises at least two hydrogen atoms per molecule;
(2) at least one diol or a mixture of diols comprising acid functions or their salts;
(3) at least one di- or polyisocyanate; and
(4) at least one compound having a silicone backbone.

For instance, the compounds (1) may be chosen from diols, diamines, polyesterols, polyetherols, and mixtures thereof.

In one embodiment, for example, among the compounds (1) which may used, non-limiting mention may be made of linear polyethylene glycols and polypropylene glycols, such as those which are obtained by reaction of ethylene oxide or propylene oxide with water or diethylene or dipropylene glycol in the presence of sodium hydroxide as catalyst. These polyalkylene glycols generally have a molecular mass ranging from 600 to 20,000.

In another embodiment, organic compounds comprising mercapto, amino, carboxyl or hydroxyl groups may also be used. Among these, non-limiting mention may be made for instance, of polyhydroxylated compounds such as polyether diols, polyester diols, polyacetal diols, polyamide diols, polyesterpolyamide diols, poly(alkylene ether) diols, polythioether diols and polycarbonate diols.

Among the polyether diols, for example, non-limiting mention may be made of the condensation products of ethylene oxide, of propylene oxide or of tetrahydrofuran, their copolymerization or condensation products, which may be grafted or blocks, such as mixtures of condensates of ethylene oxide and propylene oxide, and the products of polymerization of olefins, at high pressure, with alkylene oxide condensates. Suitable polyethers include, for example, those prepared by condensation of alkylene oxides and polyhydric alcohols, such as ethylene glycol, 1,2-propylene glycol and 1,4-butanediol.

The polyester diols, polyesteramides, and polyamide diols may be, for example, saturated and can be obtained, for example, from the reaction of saturated or unsaturated polycarboxylic acids with polyhydric alcohols, diamines or polyamines. Adipic acid, succinic acid, phthalic acid, terephthalic acid and maleic acid can be used, for example, to prepare these compounds. Polyhydric alcohols that are suitable for preparing the polyesters include, for example, ethylene glycol, 1,2-propylene glycol, 1,4-butanediol, neopentyl glycol and hexanediol. Amino alcohols, for example ethanolamine, can also be used. Diamines which are suitable for preparing the polyesteramides include ethylenediamine and hexamethylenediamine.

Suitable polyacetals can be prepared, for example, from 1,4-butanediol or from hexanediol and from formaldehyde. Suitable polythioethers can be prepared, for example, by condensation reaction between thioglycols, alone or in combination with other glycols such as ethylene glycol or 1,2-propylene glycol or with other polyhydroxylated compounds. Polyhydroxylated compounds comprising urethane groups, natural polyols, which can be further modified, for example, castor oil and carbohydrates, can also be used.

In one embodiment, the compound of group (1) may be a polyesterol, such as a polyester diol formed by the reaction of at least one (di)polyol (1ₐ) and at least one acid (1_{b}). The (di)polyol (1ₐ) may, for instance, be chosen from neopentyl glycol, 1,4-butanediol, hexanediol, ethylene glycol, diethylene glycol, propylene glycol, butylene glycol, neopentyl glycol and (di)polyethylene glycol. The acid (1_{b}) may, for instance, be chosen from phthalic acid, isophthalic acid, adipic acid and (poly)lactic acid.

A hydroxycarboxylic acid such as dimethylolpropanoic acid (DMPA) or a 2,2-hydroxymethylcarboxylic acid can, for example, be used as compound (2). In general, compound (2) is useful as a coupling block. In one embodiment of the present disclosure, the compounds (2) are those comprising at least one poly((alpha-hydroxydiolcarboxylic) acid). In another embodiment of the present disclosure, the compounds (2) are chosen from 2,2-di(hydroxymethyl)acetic acid, 2,2-dihydroxymethylpropionic acid, 2,2-dihydroxymethylbutyric acid and 2,2-dihydroxymethylpentanoic acid.

The di- or polyisocyanate (3) can be chosen for example, from hexamethylene diisocyanate, isophorone diisocyanate (IPDI), tolylene diisocyanate, 4,4′-diphenylmethane diisocyanate (DPMD), 4,4′-dicyclohexylmethane diisocyanate (DCMD), methylenebis(p-phenyl) diisocyanate, methylenebis(4-cyclohexyl isocyanate), toluene diisocyanates, 1,5-naphthalene diisocyanate, 4,4′-diphenylmethane diisocyanate, 2,2′-dimethyl-4,4′-diphenylmethane diisocyanate, 1,3-phenylene diisocyanate, 1,4-phenylene diisocyanate, mixtures of 2,4- and 2,6-toluene diisocyanates, 2,2′-dichloro-4,4′-diisocyanatodiphenylmethane, 2,4-dibromo-1,5-diisocyanatonaphthalene, 1,4-butane diisocyanate, 1,6-hexane diisocyanate and 1,4-cyclohexane diisocyanate.

The compound having a silicone backbone (4) can be chosen from polysiloxanes, polyalkylsiloxanes and polyarylsiloxanes, for instance, polyethylsiloxanes, polymethylsiloxanes and polyphenylsiloxanes, optionally comprising hydrocarbon-based chains grafted onto the silicon atoms.

The fixing polyurethane can be formed using an additional compound (5) that generally serves to extend its chain. These compounds (5) can be chosen from for example, saturated and unsaturated glycols such as ethylene glycol, diethylene glycol, neopentyl glycol and triethylene glycol; amino alcohols such as ethanolamine, propanolamine and butanolamine; heterocyclic, aromatic, cycloaliphatic and aliphatic primary amines; diamines; carboxylic acids such as aliphatic, aromatic and heterocyclic carboxylic acids, for instance oxalic acid, succinic acid, glutaric acid, adipic acid, sebacic acid and terephthalic acid; and amino carboxylic acids. In one embodiment of the present disclosure, the compounds (5) are aliphatic diols.

The at least one silicone fixing polyurethane used in the present disclosure can, for example, comprise at least one polysiloxane sequencewith a repeating base unit that corresponds, for example, to formula (II):
—O—P—O—CO—NH—R—NH—CO— (II)
wherein:
P is a polysiloxane segment, and
R is a divalent group chosen from optionally substituted alkylene groups that can be aromatic, C₁ to C₂₀ aliphatic and C₁ to C₂₀ cycloaliphatic.

By way of non-limiting example, the polysiloxane segment P may correspond to formula (III): 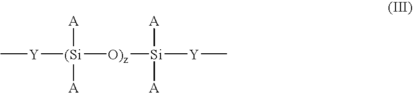
wherein:
A, which may be identical or different, is chosen from C₁ to C₂₀ monovalent hydrocarbon-based groups that are free or substantially free of ethylenic unsaturation and from aromatic groups,
Y is chosen from divalent hydrocarbon-based groups, and
z is an integer chosen such that the average molecular mass of the polysiloxane segment ranges from 300 to 10,000.

For instance, the divalent group Y may be chosen from alkylene groups of formula —(CH₂)ₐ—, in which “a” is an integer ranging from 1 to 10.

The groups A can be chosen from alkyl groups, such as methyl, ethyl, propyl, isopropyl, butyl, pentyl, hexyl, octyl, decyl, dodecyl and octadecyl groups; cycloalkyl groups, such as cyclohexyl groups; aryl groups, for instance phenyl and naphthyl; arylalkyl groups, such as benzyl, phenylethyl, tolyl and xylyl groups.

By way of examples of silicone fixing polyurethane, non-limiting mention may be made, for example, of the dimethylolpropionic acid/isophorone diiso-cyanate/neopentyl glycol/silicone-comprising polyesterdiols/diamine copolymer (also known as polyurethane-6, INCI name) sold under the trademark Luviset® Si PUR by the company BASF.

The at least one silicone fixing polyurethane may be present in the composition in an amount ranging from 0.01 to 20% by weight, such as from 0.05 to 15%, and for instance, from 0.1 and 10% by weight, relative to the total weight of the composition.

The compositions according to the present disclosure can also comprise at least one additional fixing polymer different from the silicone fixing polyurethanes described above. The at least one additional fixing polymer that are suitable in the present disclosure include those chosen from cationic, anionic, amphoteric and nonionic polymers.

The cationic additional fixing polymers which can be used according to the present disclosure may be chosen from, for example, polymers comprising primary, secondary, tertiary and/or quaternary amine groups which are part of the polymer chain or directly connected thereto, and which have a number-average molecular mass ranging from 500 to 5,000,000, such as from 1,000 to 3,000,000.

Among these polymers, non-limiting mention may be made, for example, of the following cationic polymers:
(1) homopolymers or copolymers of acrylic or methacrylic esters or amides, comprising amino functions, comprising at least one of the units of formulae (A), (B), and (C): 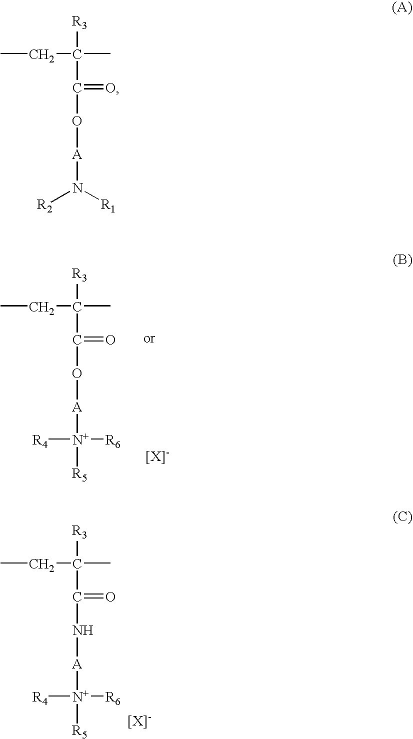
   wherein:
R₁ and R₂, which may be identical or different, are chosen from hydrogen atoms and alkyl groups having from 1 to 6 carbon atoms;
R₃ is chosen from hydrogen atoms and CH₃ groups;
A is chosen from linear and branched alkyl groups comprising from 1 to 6 carbon atoms and hydroxyalkyl groups comprising from 1 to 4 carbon atoms;
R₄, R₅ and R₆, which may be identical or different, are chosen from alkyl groups having from 1 to 18 carbon atoms and benzyl groups;
X is chosen from a methosulphate anion and halides, such as chloride or bromide.

The copolymers of family (1) also comprise at least one unit derived from comonomers that can be chosen from acrylamides, methacrylamides, diacetone acrylamides, acrylamides and methacrylamides substituted on the nitrogen with lower (C₁₋₄) alkyl groups, groups derived from acrylic or methacrylic acids or esters thereof, vinyllactams such as vinylpyrrolidone or vinylcaprolactam, and vinyl esters.

Among the copolymers of family (1), non-limiting mention may be made of:
copolymers of acrylamide and of dimethylaminoethyl methacrylate, quaternized with dimethyl sulphate or with a dimethyl halide, such as the product sold under the name Hercofloc® by the company Hercules,
copolymers of acrylamide and of methacryloyloxyethyltrimethylammonium chloride described, for example, in the patent application EP-A-08 09 76 and sold under the name BINA QUAT P 100 by the company Ciba Geigy,
copolymers of acrylamide and of methacryloyloxyethyltrimethylammonium methosulphate, such as the product sold under the name Reten by the company Hercules,
quaternized or nonquaternized vinylpyrrolidone/dialkylaminoalkyl acrylate or methacrylate copolymers, such as the products sold under the names “Gafquat®” by the company ISP, such as, for example, “Gafquat® 734” or “Gafquat® 755,” or alternatively the products called “Copolymer® 845, 958 and 937;” these polymers are described in detail in French Patent Nos. 2,077,143 and 2,393,573,
polymers comprising a fatty chain and a vinylpyrrolidone unit, such as the product sold under the name STYLEZE W20 and STYLEZE W10 by the company ISP,
dimethylaminoethyl methacrylate/vinylcaprolactam/vinylpyrrolidone terpolymers, such as the product sold under the name Gaffix® VC 713 by the company ISP, and
quaternized vinylpyrrolidone/dimethylaminopropyl methacrylamide copolymers such as, for instance, the product sold under the name “Gafquat® HS 100” by the company ISP;
(2) cationic polysaccharides, for instance optionally comprising quaternary ammonium, such as those described in U.S. Pat. Nos. 3,589,578 and 4,031,307, for example, guar gums comprising trialkylammonium cationic groups. Such products are sold, for instance, under the trade names JAGUAR C13 S, JAGUAR C 15 and JAGUAR C 17 by the company Meyhall;
(3) quaternized copolymers of vinylpyrrolidone and vinylimidazole;
(4) chitosans or their salts; the salts which can be used include, for example, the acetate, lactate, glutamate, gluconate or pyrrolidonecarboxylate of chitosan.

Among these compounds, non-limiting mention may be made of the chitosan having a degree of deacetylation of 90.5% by weight, sold under the name KYTAN BRUT STANDARD by the company Aber Technologies, and the chitosan pyrrolidonecarboxylate sold under the name Kytamer® PC by the company Amerchol;
(5) cationic cellulose derivatives such as the copolymers of cellulose or of cellulose derivatives grafted with a water-soluble quaternary ammonium monomer, and described, for example, in U.S. Pat. No. 4,131,576, such as hydroxyalkylcelluloses, for instance hydroxymethyl-, hydroxyethyl- or hydroxypropyl celluloses grafted for example with a methacryloyloxyethyltrimethylammonium, methacrylamidopropyltrimethylammonium or dimethyldiallylammonium salt.

The marketed products corresponding to this definition include the products sold under the names “CELQUAT L 200” and “CELQUAT H 100” by the company National Starch.

The anionic additional fixing polymers that may be used are polymers comprising groups derived from carboxylic, sulphonic or phosphoric acid, and have a number-average molecular mass ranging from 500 to 5,000,000.

The carboxylic groups are provided by unsaturated monocarboxylic and dicarboxylic acid monomers such as those corresponding to the formula (I): 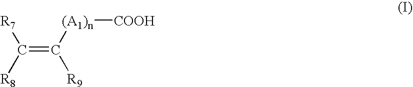
wherein n is an integer ranging from 0 to 10, A₁ is a methylene group optionally linked to the carbon atom of the unsaturated group, or to the neighboring methylene group when n is greater than 1, by a hetero atom such as oxygen or sulphur, R₇ is chosen from a hydrogen atom, and phenyl and benzyl groups, R₈ is chosen from a hydrogen atom, and lower alkyl and carboxyl groups, R₉ is chosen from a hydrogen atom, lower alkyl groups, —CH₂—COOH groups, phenyl groups and benzyl groups.

In the context of the abovementioned formula (I), a lower alkyl group may be a group having 1 to 4 carbon atoms, such as methyl and ethyl groups.

Among the anionic fixing polymers comprising carboxylic groups that may be used according to the present disclosure, non-limiting mention made be made of:

A) homopolymers or copolymers of acrylic or methacrylic acid or their salts, for instance, the products sold under the names Versicol® E or K by the company Allied Colloid, and Ultrahold® by the company BASF, copolymers of acrylic acid and of acrylamide sold in the form of their sodium salts, under the names RETEN 421, 423 or 425 by the company Hercules, sodium salts of polyhydroxycarboxylic acids.

B) Copolymers of acrylic or methacrylic acid with a monoethylenic monomer such as ethylene, styrene, vinyl esters, esters of acrylic or methacrylic acid, optionally grafted with a polyalkylene glycol such as polyethylene glycol, and optionally crosslinked. Such polymers are described for example, in French Patent No.1,222,944 and the German patent application No.2,330,956; the copolymers of this type comprise in their chain an optionally N-alkylated and/or hydroxyalkylated acrylamide unit, as described for instance in the Luxembourg patent application Nos. 75370 and 75371, or proposed under the name QUADRAMER by the company American Cyanamid. Non-limiting mention may also be made of copolymers of acrylic acid and of C₁-C₄ alkyl methacrylate and terpolymers of vinylpyrrolidone, of acrylic acid and of C₁-C₂₀ alkyl, for example lauryl, methacrylate, such as the product sold by the company ISP under the name Acrylidone® LM and methacrylic acid/ethyl acrylate/tert-butyl acrylate terpolymers such as the product sold under the name Luvimer® 100 P by the company BASF.

Further non-limiting mention may also be made of the methacrylic acid/acrylic acid/ethyl acrylate/methyl methacrylate copolymers in aqueous dispersion, sold under the name Amerhold® DR 25 by the company Amerchol.

C) Crotonic acid copolymers, such as those comprising in their chain vinyl acetate or propionate units, and optionally other monomers such as the allyl or methallyl esters, vinyl ether or vinyl ester of a linear or branched, saturated carboxylic acid comprising a long hydrocarbon-based chain, for instance those comprising at least 5 carbon atoms, it being possible for these polymers to be optionally grafted or crosslinked, or else another monomer of a vinyl, allyl or methallyl ester of an α- or β-cyclic carboxylic acid. Such polymers are described, inter alia, in French Patent Nos. 1,222,944, 1,580,545, 2,265,782, 2,265,781, 1,564,110 and 2,439,798. Commercial products which fall into this class include the resins 28-29-30, 26-13-14 and 28-13-10 sold by the company National Starch.

D) Copolymers of monounsaturated C₄-C₈ carboxylic acids or anhydrides, chosen from:
copolymers comprising (i) at least one entity chosen from maleic, fumaric and itaconic acids and anhydrides and (ii) at least one monomer chosen from vinyl esters, vinyl ethers, vinyl halides, phenylvinyl derivatives, acrylic acid and its esters, wherein the anhydride functional groups of these polymers are optionally monoesterified or monoamidated. Such polymers are described, for instance, in U.S. Pat. Nos. 2,047,398, 2,723,248 and 2,102,113, and GB Patent No. 839,805. Commercial products include, for example, those sold under the names Gantrez® AN or ES by the company ISP; and
copolymers comprising (i) at least one unit chosen from maleic, citraconic and itaconic anhydride units and (ii) at least one monomer chosen from allyl or methallyl esters optionally comprising in their chain at least one group chosen from acrylamide, methacrylamide, a-olefin, acrylic ester, methacrylic ester, acrylic acid, methacrylic acid, and vinylpyrrolidone groups,
wherein the anhydride functional groups of these polymers are optionally monoesterified or monoamidated. These polymers are, for example, described in French Patent Nos. 2,350,384 and 2,357241.

E) Polyacrylamides comprising carboxylate groups.

The homopolymers and copolymers comprising sulphonic groups are polymers comprising vinylsulphonic, styrenesulphonic, naphthalenesulphonic or acrylamido-alkylsulphonic units.

By way of non-limiting example, these polymers can be, for example, chosen from:
the salts of polyvinylsulphonic acid having a molecular mass ranging from 1,000 to 100,000, and also the copolymers with an unsaturated comonomer such as acrylic or methacrylic acids and their esters, and acrylamide or its derivatives, vinylethers and vinylpyrrolidone;
the salts of polystyrenesulphonic acid such as the sodium salts sold, for example, under the names Flexan® 500 and Flexan® 130 by National Starch. These compounds are described in French Patent No. 2,198,719;
the salts of polyacrylamidesulphonic acids such as those mentioned in U.S. Pat. No. 4,128,631, for instance, the polyacrylamidoethylpropanesulphonic acid sold under the name COSMEDIA POLYMER HSP 1180 by Henkel.

As another anionic additional fixing polymer which can be used according to the present disclosure, non-limiting mention may be made of the anionic branched block polymer sold under the name FIXATE G100 by the company Noveon.

According to the present disclosure, the anionic additional fixing polymers may also be chosen from, for example, acrylic acid copolymers such as the acrylic acid/ethyl acrylate/N-tert-butylacrylamide terpolymers sold for instance under the name Ultrahold® Strong by the company BASF, copolymers derived from crotonic acid such as the vinyl acetate/vinyl tert-butylbenzoate/crotonic acid terpolymers and the crotonic acid/vinyl acetate/vinyl neododecanoate terpolymers sold, for example, under the name RÉSINE 28-29-30 by the company National Starch, polymers derived from maleic, fumaric or itaconic acids or anhydrides with vinyl esters, vinyl ethers, vinyl halides, phenylvinyl derivatives, acrylic acid and its esters, such as the methyl vinyl ether/monoesterified maleic anhydride copolymers sold, for example, under the name Gantrez® by the company ISP, the copolymers of methacrylic acid and of methyl methacrylate sold under the name Eudragit® L by the company Rohm Pharma, the copolymers of methacrylic acid and of ethyl acrylate sold under the name Luvimer® MAEX or MAE by the company BASF, vinyl acetate/crotonic acid copolymers such as those sold under the name Luviset® CA 66 by the company BASF, the vinyl acetate/crotonic acid copolymers grafted with polyethylene glycol sold under the name Aristoflex® A by the company BASF, and the polymer sold under the name FIXATE G100 by the company Noveon.

Among the anionic additional fixing polymers mentioned above, non-limiting mention may be made of the monoesterified methyl vinyl ether/maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers, the crotonic acid/vinylacetate/vinyl neododecanoate terpolymers sold under the name RÉSINE 28-29-30 by the company National Starch, the copolymers of methacrylic acid and of ethyl acrylate sold under the name Luvimer® MAEX or MAE by the company BASF, the vinylpyrrolidone/acrylic acid/lauryl methacrylate terpolymers sold under the name Acrylidone® LM by the company ISP, and the polymer sold under the name FIXATE G100 by the company Noveon.

The amphoteric additional fixing polymers that can be used in accordance with the present disclosure can be chosen from polymers comprising B and C units distributed randomly in the polymer chain, wherein B is a unit derived from a monomer comprising at least one basic nitrogen atom and C is a unit derived from an acid monomer comprising at least one group chosen from carboxylic and sulphonic groups, or alternatively, B and C can be groups derived from zwitterionic monomers of carboxybetaines or of sulphobetaines.

B and C can also be a cationic polymer chain comprising primary, secondary, tertiary or quaternary amine groups, in which at least one of the amine groups bears a carboxylic or sulphonic group linked via a hydrocarbon-based group, or alternatively, B and C are part of a chain of a polymer comprising ethylene-α,β-dicarboxylic units, one of the carboxylic groups of which has been brought to react with a polyamine comprising at least one primary or secondary amine group.

Among the amphoteric fixing polymers that correspond to the definition given above, non-limiting examples include the following polymers:
(1) copolymers comprising acidic vinyl units and basic vinyl units, such as those resulting from the copolymerization of a monomer derived from a vinyl compound bearing a carboxylic group such as, for example, acrylic acid, methacrylic acid, maleic acid or alpha-chloroacrylic acid, and of a basic monomer derived from a substituted vinyl compound comprising at least one basic atom, such as, for instance, dialkylaminoalkyl methacrylate, acrylate, dialkylaminoalkylmethacrylamide and -acrylamide. Such compounds are described in U.S. Pat. No. 3,836,537;
(2) polymers comprising units derived:
   a) from at least one monomer chosen from acrylamides and methacrylamides substituted on the nitrogen atom with an alkyl group,
   b) from at least one acidic comonomer comprising at least one reactive carboxylic group, and
   c) from at least one basic comonomer such as acrylic or methacrylic acid esters comprising primary, secondary, tertiary and quaternary amine substituents, and the product of quaternization of dimethylaminoethyl methacrylate with dimethyl or diethyl sulphate.

The N-substituted acrylamides or methacrylamides according to the present disclosure may be, for example, the compounds in which the alkyl groups comprise from 2 to 12 carbon atoms, such as N-ethylacrylamide, N-tert-butylacrylamide, N-tert-octylacrylamide, N-octylacrylamide, N-decylacrylamide and N-dodecylacrylamide, and also the corrresponding methacrylamides.

By way of non-limiting example, the acidic comonomers may be chosen from, for example, acrylic acid, methacrylic acid, crotonic acid, itaconic acid, maleic acid, fumaric acid, and also the alkyl monoesters, having from 1 to 4 carbon atoms, of maleic and fumaric acids and anhydrides.

In one embodiment of the present disclosure, the basic comonomers are aminoethyl, butylaminoethyl, N,N′-dimethylaminoethyl and N-tert-butylaminoethyl methacrylates.

In another embodiment of the present disclosure, the copolymers whose CTFA (4th edition, 1991) name is octylacrylamide/acrylates/butylaminoethyl methacrylate copolymer, such as the products sold under the name Amphomer® or Lovocryl® 47 by the company National Starch, are used;
(3) crosslinked and acylated polyamino amides partially or totally derived from polyamino amides of formula (VII):
   CO—R₁₀—CO-Z (VII)
   wherein R₁₀ is a divalent group chosen from the derivatives of saturated dicarboxylic acids, monocarboxylic aliphatic acids, dicarboxylic aliphatic acids containing an ethylenic double bond, an ester of a lower alkanol having 1 to 6 carbon atoms, of these acids, or a group derived from the addition of any one of said acids to a bis(primary) or bis(secondary) amine, and wherein Z is chosen from derivatives of bis(primary), mono- and bis(secondary) polyalkylene-polyamine groups, and can be, by way of non-limiting example:
a) present in an amount ranging from 60 to 100 mol %, the formula (V III): 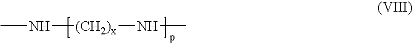
   wherein x is equal to 2, and p is either 2 or 3, or alternatively x is equal to 3 and p is equal to 2,
   formula (VII) being derived from diethylenetriamine, from triethylenetetraamine or from dipropylenetriamine;
b) present in an amount ranging from 0 to 40 mol % of formula (VIII) above, wherein x is equal to 2 and p is equal to 1 and which is derived from ethylenediamine, or the group derived from piperazine: 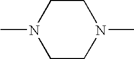
c) present in an amount ranging from 0 to 20 mol %, the group —NH—(CH₂)₆—NH-derived from hexamethylenediamine,
   these polyamino amides being crosslinked by reaction addition of a difunctional crosslinking agent chosen from epihalohydrins, diepoxides, dianhydrides and bis-unsaturated derivatives, using from 0.025 to 0.35 mol of crosslinking agent per amine group of the polyamino amide, and acylated by the action of acrylic acid, chloroacetic acid, an alkane sultone, or the salts thereof.

The saturated carboxylic acids may be chosen from, for example, acids having 6 to 10 carbon atoms, such as adipic acid, 2,2,4-trimethyladipic acid, and 2,4,4-trimethyladipic acid; terephthalic acid; and acids containing an ethylenic double bond, for instance acrylic acid, methacrylic acid and itaconic acid.

The alkane sultones used in the acylation may be, for example, propane sultone or butane sultone, and the salts of the acylating agents may be, for instance sodium or potassium salts;
(4) polymers comprising zwitterionic units of formula (IV): 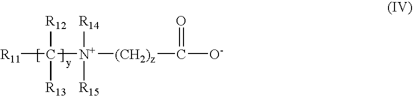
   wherein R₁₁ is a polymerizable unsaturated group such as an acrylate, methacrylate, acrylamide or methacrylamide group, y and z are integers ranging from 1 to 3, R₁₂ and R₁₃ are chosen from hydrogen atoms, and methyl, ethyl and propyl groups, R₁₄ and R₁₅ are chosen from hydrogen atoms and alkyl groups with the proviso that the sum of the carbon atoms in R₁₄ and R₁₅ does not exceed 10. The polymers comprising such units can also comprise units derived from nonzwitterionic monomers such as dimethyl- or diethylaminoethyl acrylates, methacrylates, alkyl acrylates, alkylmethacrylates, acrylamides, methacrylamides, or vinyl acetate.

By way of example, non-limiting mention may be made of methyl methacrylate/methyl dimethylcarboxymethylammonioethylmethacrylate copolymers, such as the product sold under the name DIAFORMER Z301 by the company Sandoz;
(5) polymers derived from chitosan comprising monomer units corresponding to formulae (D), (E), and (F): 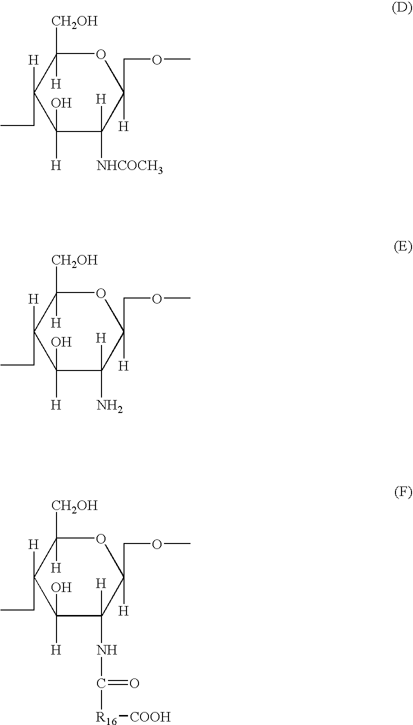
   wherein the unit (D) is present in an amount ranging from 0 to 30%, the unit (E) is present in an amount ranging from 5 to 50% and the unit (F) is present in an amount ranging from 30 to 90%, it being understood that, in this unit (F), R₁₆ is a group of formula: 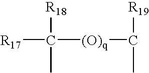
   wherein, if q is equal to 0, R₁₇, R₁₈ and R₁₉, which may be identical or different, are chosen from hydrogen atoms, methyl, hydroxyl, acetoxy amino, monoalkylamine, and dialkylamine groups, optionally interrupted by at least one nitrogen atom and/or optionally substituted with at least one group chosen from amine, hydroxyl, carboxyl, alkylthio and sulphonic groups, and alkylthio groups in which the alkyl group bears an amino, with the proviso that in such an instance, at least one of R₁₇, R₁₈ and R₁₉ is a hydrogen atom;
or, if q is equal to 1, then R₁₇, R₁₈ and R₁₉ are each a hydrogen atom, and also the salts formed by these compounds with bases or acids;
(6) polymers corresponding to formula (V) which are, for example, described in French Patent No. 1,400,366: 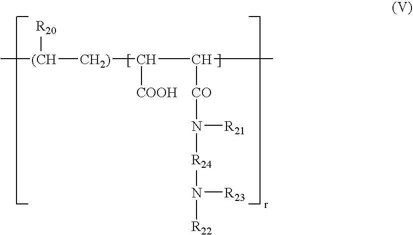
   wherein R₂₀ is chosen from a hydrogen atom, and CH₃O, CH₃CH₂O, and phenyl groups, R₂₁ is chosen from a hydrogen atom and lower alkyl groups such as methyl or ethyl, R₂₂ is chosen from a hydrogen atom and C₁-C₆ lower alkyl groups such as methyl or ethyl, R₂₃ is chosen from C₁-C₆ lower alkyl groups such as methyl or ethyl, and —R₂₄—N(R₂₂)₂ groups, R₂₄ is chosen from —CH₂—CH₂—, —CH₂—CH₂—CH₂— and —CH₂—CH(CH₃)— groups, R₂₂ being defined as above;
(7) polymers derived from the N-carboxyalkylation of chitosan, such as N-carboxymethylchitosan or N-carboxybutylchitosan, sold under the name “EVALSAN” by the company Jan Dekker;
(8) amphoteric polymers of the type -D-X-D-X chosen from:
   a) polymers obtained by the action of chloroacetic acid or sodium chloroacetate on compounds comprising at least one unit of formula:
      -D-X-D-X-D- (VI)
      wherein D is a group 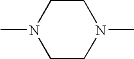
      and X is chosen from E and E′, wherein E and E′, which may be identical or different, are divalent groups chosen from straight and branched chain alkylene groups comprising up to 7 carbon atoms in the main chain, optionally substituted with hydroxyl groups and which can comprise oxygen, nitrogen and sulphur atoms, and 1 to 3 aromatic and/or heterocyclic rings; wherein the oxygen, nitrogen and sulphur atoms are present in the form of ether, thioether, sulphoxide, sulphone, sulphonium, alkylamine, alkenylamine, hydroxyl, benzylamine, amine oxide, quaternary amine, amide, imide, alcohol, ester and/or urethane groups;
b) polymers of formula (VI′):
   -D-X-D-X- (VI′)
   wherein D is a group: 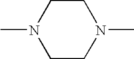
   and X is chosen from E and E′, with the proviso that X is at least once E′; wherein E is as defined above and E′ is a divalent group chosen from straight and branched chain alkylene groups comprising up to 7 carbon atoms in the main chain, optionally substituted with at least one hydroxyl group and comprising at least one nitrogen atom, the nitrogen atom being substituted with an alkyl chain that is optionally interrupted by an oxygen atom and necessarily comprises at least one functional group chosen from carboxyl and hydroxyl functional groups, betainized by reaction with chloroacetic acid or sodium chloroacetate;
(9) (C₁-C₅)alkyl vinyl ether/maleic anhydride copolymers partially modified by semiamidation with an N,N-dialkylaminoalkylamine such as N,N-dimethylamino-propylamine, or by semiesterification with an N,N-dialkylaminoalkanol. These copolymers can also contain other vinyl comonomers such as vinylcaprolactam.

Among the amphoteric additional fixing polymers described above that may be used according to the present disclosure, non-limiting mention may be made of those of family (3), such as copolymers whose CTFA name is octylacrylamide/acrylates/ butylaminoethyl methacrylate copolymer, such as the products sold under the names Amphomer®, Amphomer® LV 71 or Lovocryl® 47 by the company National Starch and those of family (4), such as methyl methacrylate/methyl dimethylcarboxymethyl-ammonioethylmethacrylate copolymers sold, for example, under the name Diaformer® Z301 by the company Sandoz.

The nonionic additional fixing polymers that can be used according to the present disclosure are chosen, for example, from:
polyalkyloxazolines;
vinyl acetate homopolymers;
vinyl acetate copolymers such as, for example, copolymers of vinyl acetate and of acrylic ester, copolymers of vinyl acetate and of ethylene, or copolymers of vinyl acetate and of maleic ester, for example of dibutyl maleate;
homopolymers and copolymers of acrylic esters, for instance copolymers of alkyl acrylates and of alkyl methacrylates, such as the products proposed by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl (meth)acrylates; non-limiting mention may be made of the products provided under the name CJ 0601 B by the company Rohm & Haas;
styrene homopolymers;
styrene copolymers, for instance copolymers of styrene and of alkyl (meth)acrylate, such as the products Mowilith® LDM 691 1, Mowilith® DM 611 and Mowilith® LDM 6070 provided by the company Hoechst, the products Rhodopas® SD 215 and Rhodopas® DS 910 provided by the company Rhone Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers different from the vinylpyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly-(vinylpyrrolidone/vinyl acetate) copolymers such as those sold under the name PVPVA® S630L by the company ISP, and Luviskol® VA 73, VA 64, VA 55, VA 37 and VA 28 by the company BASF; and poly(vinylpyrrolidone/vinyl acetate/vinyl propionate) terpolymers such as, for example, the product sold under the name Luviskol® VAP 343 by the company BASF.

In one embodiment of the present disclosure, the alkyl groups in the nonionic polymers mentioned above have from 1 to 6 carbon atoms.

According to the present disclosure, use may also be made of additional fixing polymers of grafted silicone type, comprising a polysiloxane portion and a portion comprising a nonsilicone organic chain, one of the two portions constituting the main chain of the polymer and the other being grafted onto said main chain. These polymers are, for example, described in patent applications EP-A-0 412 704, EP-A-0 412 707, EP-A-0 640 105 and WO 95/00578, EP-A-0 582 152 and WO 93/23009 and U.S. Pat. Nos. 4,693,935; 4,723,571 and 4,972,037. These polymers may be chosen from amphoteric, anionic and nonionic polymers, such as anionic or nonionic polymers.

Such polymers are, for example, the copolymers which can be obtained by free-radical polymerization using the mixture of monomers made up of:
a) from 50 to 90% by weight of tert-butyl acrylate,
b) from 0 to 40% by weight of acrylic acid,
c) from 5 to 40% by weight of a silicone macromer of formula 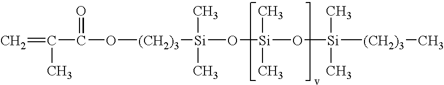
   wherein v is a number ranging from 5 to 700, the percentages by weight being calculated relative to the total weight of the monomers.

Other non-limiting examples of grafted silicone polymers include polydimethylsiloxanes (PDMSs) onto which are grafted, via a thiopropylene-type connecting chain, mixed polymer units of the poly((meth)acrylic acid) type and of the poly(alkyl (meth)acrylate) type, and polydimethylsiloxanes (PDMSs) onto which are grafted, via a thiopropylene-type connecting chain, polymer units of the isobutyl poly(meth)acrylate type. Additional fixing polymers that may also be used include functionalized or nonfunctionalized, nonsilicone, cationic, nonionic, anionic or amphoteric polyurethanes, or mixtures thereof.

Among the polyurethanes that may be used according to the present disclosure are those described in applications EP 0 751 162, EP 0 637 600, EP 0 648 485 and FR 2,743,297, from the company L'Oreal, and also in applications EP 0 656 021 and WO 94/03510 from the company BASF, and EP 0 619 111 from the company National Starch.

Among other polyurethanes which may be suitable in the present disclosure, non-limiting mention may be made of the product sold under the name Luviset Pur® by the company BASF.

The cosmetically acceptable medium comprises, for example, water, or at least one cosmetically acceptable solvent, such as alcohols, or water-solvent(s) mixtures. In one embodiment of the present disclosure, the solvents are chosen from C₁-C₄ alcohols,
such as ethanol and isopropanol. In another embodiment of the present disclosure, the alcohol is ethanol.

The composition as disclosed herein can also contain at least one additive chosen from nonionic, anionic, cationic, amphoteric and zwitterionic surfactants; fragrances; screening agents; preserving agents; proteins; vitamins; nonionic, anionic, cationic, amphoteric and zwitterionic polymers other than the fixing polymers described above; mineral, plant and synthetic oils; and any other additive conventionally used in cosmetic compositions, such as anti-dandruff agents, anti-hairloss agents, dyes, pigments, moisturizers such as glycerol and the like, polyols, and reducing agents.

These additives may be present in the composition in an amount ranging from 0.001 to 20% by weight of the total weight of the composition. The precise amount of each additive depends on its nature and is readily determined by those skilled in the art, and will depend on the chosen hair application. Of course, those skilled in the art will take care to choose the additive(s) in such a way that the advantageous properties of the composition in accordance with the present disclosure are not, or are not substantially, altered by the envisaged addition.

The compositions of the invention can be used for producing many hair products, such as, for example, products for fixing and/or holding the hair, conditioning products, such as sheen formulations, or else haircare products.

These compositions can be packaged in various forms, such as pump-dispenser bottles or aerosol containers, in order to provide composition application in sprayed form or in mousse form. Such packaging forms are indicated, for example, when it is desired to obtain a spray, a lacquer, or a mousse for fixing or treating the hair. The compositions in accordance with the present disclosure can also be in the form of creams, gels, emulsions or lotions.

When the composition as disclosed herein is packaged in the form of an aerosol for the purpose of obtaining a lacquer or a mousse, it further comprises at least one propellant that can be chosen from volatile hydrocarbons such as n-butane, propane, isobutane, pentane, a halogenated hydrocarbon and mixtures thereof. As a propellant, use may also be made of carbon dioxide, nitrous oxide, dimethyl ether (DME), nitrogen and compressed air. Mixtures of propellants may also be used. In one embodiment of the present disclosure, the propellant is dimethyl ether.

The propellant may be present in the composition in an amount ranging from 5 to 90% by weight, relative to the total weight of the composition in the aerosol device, for instance, from 10 to 60%.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The following examples are intended to illustrate the invention in a non-limiting manner.

### EXAMPLE 1

Composition A below was applied to a lock of natural euro-chestnut hair of 5 grams.

Composition A:

| | | |
|---|---|---|
| Permethyl 102 A (1) | | 4% |
| Luviset Si Pur ® (2) | | 5% |
| Ethanol | qs | 100% |

(1) isoeicosane sold by the company Bayer
(2) Polyurethane 6 (isophthalic acid/adipic acid/hexylene glycol/neopentyl glycol/dimethylpropanoic acid/isophorone diisocyanate/bisethylaminoisobutyldimethicone copolymer), sold by the company BASF.

Composition A was applied to natural hair using a pump-dispenser bottle. After drying, it was observed that the lock had good hold, a sheen, was soft to the touch, and had a pleasant appearance.

### EXAMPLE 2

Composition B below was applied to a lock of natural euro-chestnut hair of 5 grams.

Composition B:

| | | |
|---|---|---|
| Permethyl 102 A (1) | | 10% |
| DC 245 fluid (2) | | 5% |
| DC 2-5225 C (3) | | 7% |
| Luviset Si Pur ® (4) | | 10% |
| Water | qs | 100% |

(1) isoeicosane sold by the company Bayer
(2) cyclopentadimethylsiloxane sold by the company Dow Corning
(3) oxyethylenated oxypropylenated polydimethyl/methylsiloxane sold by the company Dow Corning
(4) Polyurethane 6 (isophthalic acid/adipic acid/hexylene glycol/neopentyl glycol/dimethyloipropanoic acid/isophorone diisocyanate/bisethylaminoisobutyldimethicone copolymer), sold by the company BASF.

Composition B was applied to natural hair using a pump-dispenser bottle. After drying, it was observed that the lock had good hold, a sheen, was soft to the touch, and had a pleasant appearance.

1. A cosmetic hair composition, comprising, in a cosmetically acceptable medium, isoeicosane and at least one silicone fixing polyurethane.

2. The composition according to claim 1,wherein the isoeicosane is present in the composition in an amount ranging from 0.05 to 20% by weight, relative to the total weight of the composition.

3. The composition according to claim 2, wherein the isoeicosane is present in the composition in an amount ranging from 1 to 10% by weight, relative to the total weight of the composition.

4. The composition according to claim 1, wherein the at least one silicone fixing polyurethane is chosen from cationic, anionic, amphoteric, and nonionic silicone fixing polyurethanes.

5. The composition according to claim 1, wherein the at least one silicone fixing polyurethane comprises a repeating base unit of formula (II):
—O—P—O—CO—NH—R—NH—CO— (II)
wherein:
P is a polysiloxane segment, and
R is a divalent group chosen from optionally substituted alkylene groups that may be aromatic, C₁ to C₂₀ aliphatic and C₁ to C₂₀ cycloaliphatic.

6. The composition according to claim 5, wherein the at least one fixing polyurethane is a dimethylolpropionic acid/isophorone diisocyanate/neopentyl glycol/silicone-comprising polyesterdiols/diamine copolymer.

7. The composition according to claim 1, wherein the at least one silicone fixing polyurethane is present in the composition in an amount ranging from 0.01 to 20% by weight, relative to the total weight of the composition.

8. The composition according to claim 7, wherein the at least one silicone fixing polyurethane is present in the composition in an amount ranging from 0.05 to 15% by weight, relative to the total weight of the composition.

9. The composition according to claim 8, wherein the at least one silicone fixing polyurethane is present in the composition in an amount ranging from 0.1 to 10% by weight, relative to the total weight of the composition.

10. The composition according to claim 1, further comprising at least one additional fixing polymer.

11. The composition according to claim 1, further comprising at least one additive chosen from nonionic, anionic, cationic, amphoteric and zwitterionic surfactants; fragrances; screening agents; preserving agents; proteins; vitamins; nonionic, anionic, cationic and zwitterionic polymers different from those that may already be present; mineral, plant and synthetic oils; anti-dandruff agents; anti-hairloss agents; dyes; pigments; moisturizers; and reducing agents.

12. A cosmetic process for holding a hairstyle, comprising applying to hair a composition comprising, in a cosmetically acceptable medium and in a combined amount effective to hold a hairstyle, isoeicosane and at least one silicone fixing polyurethane.

13. The process according to claim 12, wherein the isoeicosane is present in the composition in an amount ranging from 0.05 to 20% by weight, relative to the total weight of the composition.

14. The process according to claim 12, wherein the at least one silicone fixing polyurethane is chosen from cationic, anionic, amphoteric, and nonionic silicone fixing polyurethanes.

15. The process according to claim 12, wherein the at least one silicone fixing polyurethane comprises a repeating base unit of formula (II):
—O—P—O—CO—NH—R—NH—CO— (II)
wherein:
P is a polysiloxane segment, and
R is a divalent group chosen from optionally substituted alkylene groups that may be aromatic, C₁ to C₂₀ aliphatic and C₁ to C₂₀ cycloaliphatic.

16. The process according to claim 15, wherein the at least one fixing polyurethane is a dimethylolpropionic acid/isophorone diisocyanate/neopentyl glycov/silicone-comprising polyesterdiols/diamine copolymer.

17. The process according to claim 12, wherein the at least one silicone fixing polyurethane is present in the composition in an amount ranging from 0.01 to 20% by weight, relative to the total weight of the composition.

18. The process according to claim 12, wherein the composition further comprises at least one additional fixing polymer.

19. The composition according to claim 12, wherein the composition further comprises at least one additive chosen from nonionic, anionic, cationic, amphoteric and zwitterionic surfactants; fragrances; screening agents; preserving agents; proteins; vitamins; nonionic, anionic, cationic and zwitterionic polymers different from those that may already be present; mineral, plant and synthetic oils; anti-dandruff agents; anti-hairloss agents; dyes; pigments; moisturizers; and reducing agents.

20. A cosmetic process for giving hair a sheen, comprising applying to hair a composition comprising, in a cosmetically acceptable medium and in a combined amount effective for providing a sheen to the hair, isoeicosane and at least one silicone fixing polyurethane.
